(19) United States
(12) Patent Application Publication
(10) Pub. No.: US 2022/0296854 A1
(43) Pub. Date: Sep. 22, 2022

(54) Glans Guard

(71) Applicant: Michael Chieffo, Kernersville, NC (US)

(72) Inventor: Michael Chieffo, Kernersville, NC (US)

(21) Appl. No.: 17/584,604

(22) Filed: Jan. 26, 2022

Related U.S. Application Data
(60) Provisional application No. 63/163,087, filed on Mar. 19, 2021.

(51) Int. Cl. A61M 25/01; A61M 25/00

(52) U.S. Cl. CPC A61M 25/0111; A61M 25/0017; A61M 2025/0206

(57) ABSTRACT

The present invention relates to a guard or protective device for the glans and foreskin of a male reproductive organ or penis during a urinary catheterization process. The device generally includes a tubular body designed to be worn on a male reproductive organ or penis. The tubular body includes an opening for receiving a catheter, and an open bottom end for receiving the penis. A strap attached to the tubular body is placed below the scrotum to maintain the tubular body covering the penis in a desired position for the catheterization process. The strap can be adjusted, using at least one ladder lock buckle or similar fastener.

### CROSS-REFERENCE TO RELATED APPLICATION

The present application claims priority to, and the benefit of, U.S. Provisional Application No. 63/163,087, which was filed on Mar. 19, 2021 and is incorporated herein by reference in its entirety.

### FIELD OF THE INVENTION

The present invention relates generally to the field of urinary catheterization protection device. More specifically, the present invention relates to a glans protective device for providing protection to the glans and foreskin of a penis from irritation, deterioration, and infection. The device comprising a tubular body designed to be worn over a male reproductive organ or penis, an opening at a terminal end for receiving a catheter tube, a flexible grommet circumscribing the opening, and a strap attached to the tubular body via an opposing pair of slots of the tubular body. The strap is configured to support the tubular body and is placed around the scrotum such that the scrotum lies between the strap and the tubular body. The strap can be adjusted using ladder lock buckles to fit around the scrotum of a male user. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, urinary catheterization is performed for draining urine from a patient's bladder. The urinary catheterization is also performed for injecting fluids into a patient's bladder for treatment purposes of the bladder. Generally, in urinary catheterization, a latex, polyurethane, or silicone tube known as a urinary catheter is inserted into the bladder through the urethral opening.

While performing a urinary catheterization, generally an indwelling catheter is used that is inserted into the bladder through the urethral opening. One end of the catheter is inserted into the bladder while the other end is used for draining the urine. When the catheterization process is completed, the catheter is removed.

Catheterization for male patients typically involves inserting the catheter into the urinary tract through the cavernous portion of the urethra, which runs along the length of the ventral surface of the penis. The catheter then passes through the membranous portion of the urethra and through the prostatic and pre-prostatic portions of the urethra into the bladder. Ordinarily, the length of the cavernous portion of the urethra fluctuates. When the catheterization process is performed on a male patient through the urethral opening; the urethral opening, glans, and foreskin of the penis are vulnerable to deterioration and breakdown. This leads to skin infections and severe pain. Sometimes, the pain is so severe that a patient is not able to sleep and walk. The exposed penis during the catheterization process leads to disadvantages for male patients.

Also, during the catheterization process, the desired position of the penis is not maintained, which poses difficulties during the process, and may lead to more pain and infection for the patient. Further, to maintain the penis in the desired position, a doctor or a medical practitioner needs to hold the penis during the catheterization process, which can be inconvenient for the doctor.

Therefore, there exists a long felt need in the art for a protective device for a penis that eliminates penile head and skin deterioration and irritation caused during the conventional urinary catheterization process. There is also a long felt need in the art for a protective device for the penis that protects the penis, penile head, and the glans during the conventional urinary catheterization process. Additionally, there is a long felt need in the art for a protective device for the penis that maintains a desired position of the penis during the catheterization process. Moreover, there is a long felt need in the art for a protective device for the penis that enables a medical practitioner to use both hands for the catheter, thereby enabling more precision and accuracy. Further, there is a long felt need in the art for a protective device for the penis that reduces pain and infection during and after the catheterization process. Finally, there is a long felt need in the art for a protective device for the penis that protects the urethral opening and glans of the penis during catheterization, to prevent deterioration of the skin, and offers effective pain relief to the male patient.

The subject matter disclosed and claimed herein, in one potential embodiment thereof, comprises a glans guard device that is configured to be used as a protective device for the glans of a male reproductive organ during the urinary catheterization process. More specifically, the glans guard device comprises a tubular body having a dome-shaped top end and an open bottom end, an opening at a terminal or top end for receiving a catheter tube, a flexible grommet circumscribing the opening, and a pair of two slots positioned opposite to each other. Further, a strap is slotted continuously through the opposing pair of slots to attach the strap to the tubular body, wherein the strap includes a pair of ladder lock buckles that enables the length of the strap to be adjusted. The tubular body is designed to be worn over the male reproductive organ and the strap is designed to be worn around the scrotum, by passing under the scrotum to support the tubular body. The opening is configured to receive a urinary catheter tube therethrough for performing the urinary catheterization process without deterioration of the foreskin of the male reproductive organ. The rubber grommet circumscribing the opening is sized to frictionally retain the catheter tube in place and prevent unwanted fore and aft movement.

In this manner, the novel glans guard device of the present invention accomplishes all of the forgoing objectives, and provides a relatively safe, easy and convenient solution to eliminate skin irritation and infection around the tip and glans of a male patient during and after the urinary catheterization process. The glans guard device is easy to attach and detach and maintains the desired position of the penis during the catheterization process. The device provides protection to the foreskin of the penis. With the glans guard in place, the catheter does not contact any part of the penis other than the urethral orifice or opening. The glans guard device of the present invention is also user-friendly, as it retains desired orientation and movement of the catheter tube which reduces pain during and after the catheterization process.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one potential embodiment thereof, comprises a glans guard device that is configured to be used as a protective device for the glans of a male reproductive organ during the urinary catheterization process. More specifically, the glans guard device comprises a tubular body having a dome-shaped top end and an open bottom end, an opening at a terminal or top end for receiving a catheter tube therethrough, a flexible grommet circumscribing the opening for frictionally securing the catheter tube, and a pair of two slots positioned opposite to each other. Further, a strap is slotted continuously through the opposing pair of slots to attach the strap to the tubular body, wherein the strap includes a pair of ladder lock buckles that enables the length of the strap to be adjusted. The tubular body is designed to be worn over the male reproductive organ, and the strap is designed to be worn around the scrotum by passing under the scrotum to support the tubular body. The opening is configured to receive a urinary catheter for performing the urinary catheterization process without deterioration of the foreskin of the male reproductive organ.

In a further embodiment of the present invention, a glans protective device for preventing breakdown of the foreskin of a male reproductive organ during a urinary catheterization process is disclosed. The protective guard device includes a slotted tubular body, having a first tubular portion and a second tubular portion attached to each other through a longitudinal slit, an opening at the top of the tubular portions for receiving a urinary catheter, a bottom end to receive the male reproductive organ, a pair of two slots disposed opposite to each other near the bottom end of the tubular body, wherein each pair includes an upper slot and a lower slot separated by a supporting member, a strap inserted through said pair of straps and at least one ladder lock buckle in the strap to adjust the length of the strap.

In yet another embodiment of the present invention, the tubular body is worn over the male reproductive organ and conforms to the shape and size of the male reproductive organ. The top end conforms to the glans, and an opening at the top end can be positioned over the urethral opening/orifice. The glans guard covers the glans of the penis to protect the urethral opening/orifice during catheterization to prevent deterioration of the foreskin.

In yet another embodiment of the present invention, a method of wearing a protective device for shielding the glans of a male penis during a urinary catheterization is described. The method includes: wearing a tubular body structure of the protective device over the penis such that the top end conforms to the glans; securing the strap connected to the slots of the tubular body below the scrotum such that the scrotum lies between the tubular body and strap; positioning the penis in the desired position for the catheterization process; inserting a urinary catheter inside an opening disposed at the top end of the guard to enable insertion into a tip of the penis; and removing the protective device after the catheterization process.

In yet another embodiment of the present invention, a method of wearing a glans guard device for urinary catheterization is described. The method includes: securing a strap connected to the slots of a tubular body below the scrotum up to the perineum, such that the scrotum is positioned between the tubular body and strap; wearing the tubular body structure of the protective device over the penis such that the top end conforms to the glans; adjusting the length of the strap using a ladder lock buckle to fit to the scrotum; inserting a urinary catheter inside an opening disposed at the top end to insert into a tip of the penis; and removing the glans guard device after the catheterization process.

In yet another embodiment of the present invention, the glans guard can be designed to increase comfort and securement during the catheterization process, and protects the tip of the penis during catheterization to prevent deterioration of the skin. The device can be constructed in various sizes and materials as per the preferences of users.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a glans guard device of the present invention for catheterization in accordance with the disclosed architecture;

FIG. 2 illustrates a perspective view of one potential embodiment showing the glans guard device of the present invention used for covering the male reproductive organ of a male using a tubular body with a strap assembly wrapped around the scrotum for supporting the tubular body in accordance with the disclosed architecture;

FIG. 3 illustrates a perspective view of one potential embodiment showing attachment of the strap assembly with the tubular body and ladder lock buckles of the glans guard device of the present invention for adjusting the length of the strap assembly in accordance with the disclosed architecture;

FIG. 4 illustrates a perspective view of another potential embodiment showing a male user wearing a glans guard device of the present invention in accordance with the disclosed architecture; and

FIG. 5 illustrates an enlarged view of the opening at the top of the tubular body of the glans guard of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a protective device for a penis that eliminates penile head and skin deterioration and irritation caused during the conventional urinary catheterization process. There is also a long felt need in the art for a protective device for the penis that protects the penis, penile head and the glans during the conventional urinary catheterization process. Additionally, there is a long felt need in the art for a protective device for the penis that maintains a desired position of the penis during the catheterization process. Moreover, there is a long felt need in the art for a protective device for the penis that enables a medical practitioner to use both hands for the catheter, thereby enabling more precision and accuracy. Further, there is a long felt need in the art for a protective device for the penis that reduces pain and infection during and after the catheterization process. Finally, there is a long felt need in the art for a protective device for the penis that protects the urethral opening and glans of the penis during catheterization to prevent deterioration of the skin, and offers effective pain relief to the male patient.

The present invention, in one potential embodiment, includes a novel glans protective device for preventing breakdown of the foreskin of a male reproductive organ during a urinary catheterization process. The glans protective device includes a tubular body that can be worn over the male reproductive organ and conforms to the shape and size of the male reproductive organ. The top end of the device conforms to the glans, and an opening at the top end is positioned over the urethral opening/orifice. The glans protective device further comprises an extendable strap attached to the tubular body and is designed to be worn around the scrotum by passing under the scrotum to support the tubular body. When in use, the glans protective device covers the glans of the penis to protect the urethral opening/orifice during catheterization to prevent deterioration or injury of the foreskin.

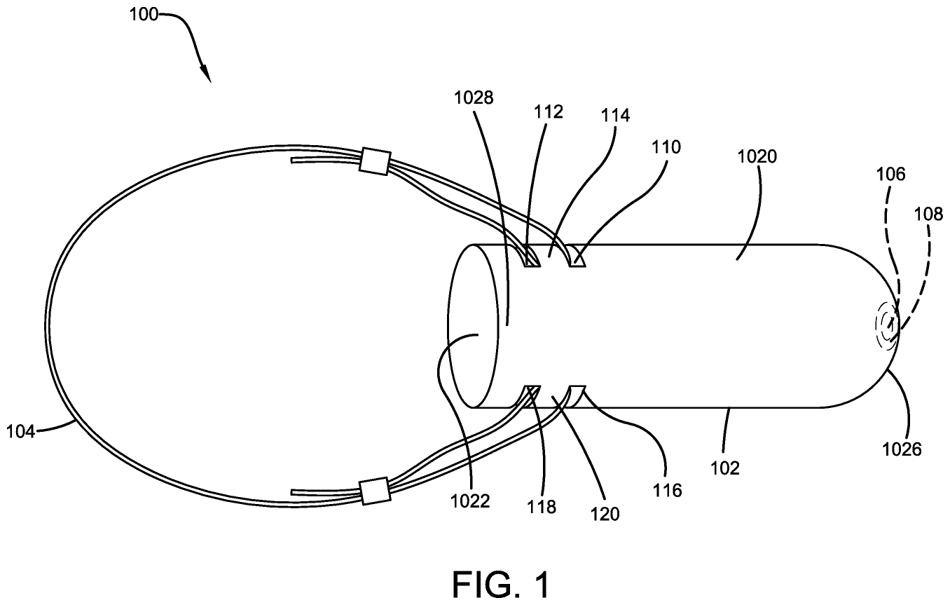

Referring to the drawings, FIG. 1 illustrates a perspective view of one potential embodiment of glans guard device of the present invention for catheterization in accordance with the disclosed architecture. The glans guard device 100 is configured to be used by medical practitioners to eliminate skin deterioration or injury of the tip of a male patient's penis during the urinary catheterization process. More specifically, the glans guard device 100 includes a tubular body 102 comprises a translucent thin and soft plastic, that is configured to be worn over the male reproductive organ or penis of a patient. The tubular body 102 includes an outer surface 1020 and an inner surface 1022. The inner surface 1022 is configured to touch the penis when the device 100 is worn. The tubular body 102 includes a dome shape at the top end 1026 to conform to the anatomy of the tip of a human penis. The top end 1026 includes a hole 106 surrounded by (i.e. circumscribed) a rubber grommet 108, wherein the hole 106 is dimensioned to receive a urinary catheter therethrough for performing the urinary catheterization process. The hole 106 is configured to receive a catheter comprising a variety of materials such as latex, polyurethane, silicone, and the like. The hole or opening 106 enables the catheter tube to pass therethrough. The grommet 108, circumscribing opening 106, is flexible and sized to frictionally retain the catheter tube and prohibit unwanted movement (i.e. fore and aft) of the catheter tube. The grommet 108 frictionally retains the catheter passing therethrough.

To provide support to the tubular body 102 during the catheterization process, an extendable strap 104 passes through a pair of two slots positioned opposite to each other in the tubular body 102 near the bottom end 1028 of the tubular body 102. A first continuous upper slot 110 and a first continuous lower slot 112, separated by a first support member 114 are positioned at a side of the tubular body 102. A second continuous upper slot 116 and a second continuous lower slot 118, separated by a second support member 120 are positioned opposite to the first continuous upper slot 110; the first continuous lower slot 112, and the first support member 114, respectively. A braided and soft elastic support strap assembly 104 passes through the continuous slots and can be positioned behind the testicles to provide support to the tubular body 102 when worn over the penis.

The braided and soft elastic strap assembly 104 includes a pair of straps fastened to each other to securely fit the tubular body 102 over the penis, wherein the penis is positioned in a desired and generally centered position within the tubular body 102. The strap assembly 104 can be extended or retracted for preventing slipping of the tubular body 102. The tubular body 102 offers effective pain relief during and after the catheterization process, and prevents skin breakdown and infections.

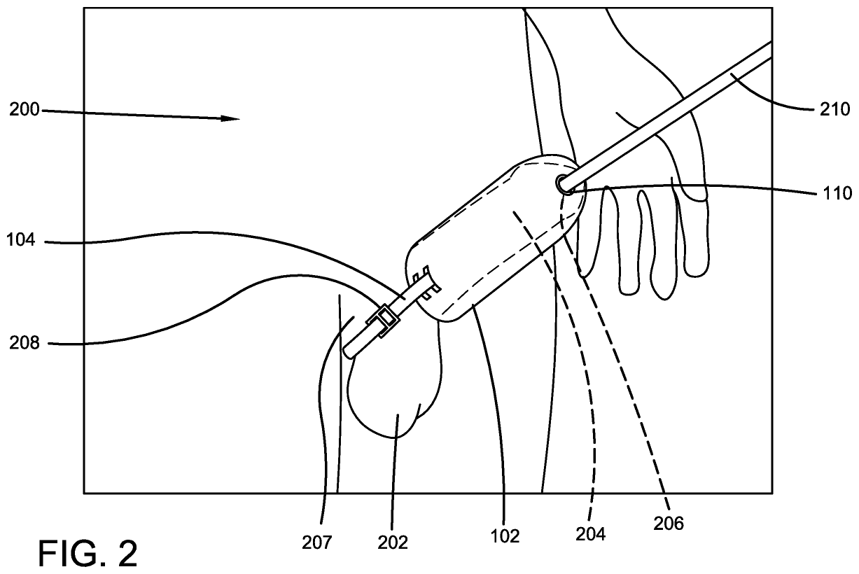

FIG. 2 illustrates a perspective view showing the glans guard device 100 of the present invention used for covering the male reproductive organ 204 of a male 200 using the tubular body 102 with the strap assembly 104 wrapped around the scrotum 202 for supporting the tubular body 102, in accordance with the disclosed architecture. To place the glans guard 100 in position, the strap assembly 104 slotted through the first continuous openings 110, 112 and second continuous openings 116, 118 is placed under the scrotum 202 (abutted against the perineum 207), such that the scrotum 202 lies between the strap assembly 104 and the tubular body 102. The strap assembly 104 can be retracted or extended to stabilize the tubular body 102 over the penis 204. The tubular body 102 conforms to the shape of the male reproductive organ 204 such that the hole 110 is positioned over the urethra orifice/opening 206 of the male reproductive organ 204. In use, the strap assembly 104 is placed under the scrotum 202, the tubular body 102 is placed over the penis 204 and the strap 104 is retracted or extended using the ladder lock buckle 208 to fit to the scrotum 202 and the male reproductive organ 204. A urinary catheter 210 can be inserted into the opening 110 disposed at the top of the tubular body 102 for guiding and inserting into the tip of the penis 204, in order to drain urine from the bladder. The glans guard 100 is useful during catheterization for urinary incontinence, urinary retention, prostate problems, and the like.

As the urinary catheter 210 is inserted into the penis 204, the guard 100 can prevent skin breakdown, and ultimately skin infections, during and after the urinary catheterization process. The tubular body 102 can be made of plastic, polycarbonate, or any other skin-friendly and lightweight material.

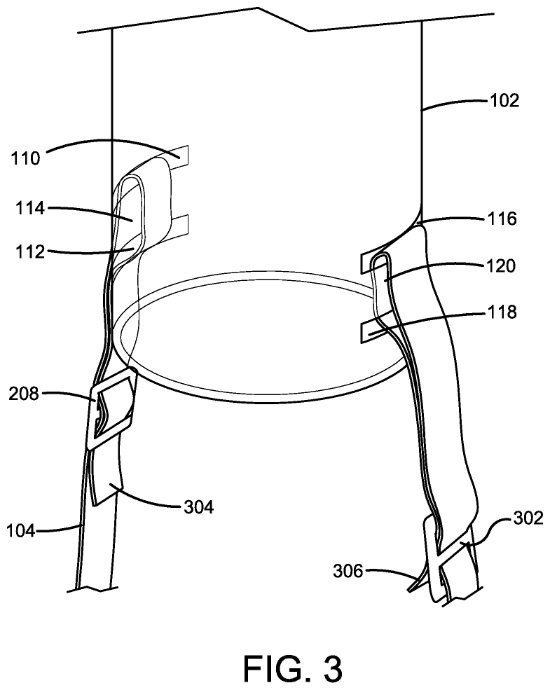

FIG. 3 illustrates a perspective view showing attachment of the strap assembly 104 with the tubular body 102 and the ladder lock buckles 208, 302 of the glans guard device of the present invention for adjusting the length of the strap assembly 104, in accordance with the disclosed architecture. The strap assembly 104 includes a pair of ladder lock buckles; a first ladder lock buckle 208 disposed near the first continuous upper slot 110 and the first continuous lower slot 112 and a second ladder lock buckle 302 disposed near the second continuous upper slot 116 and a second continuous lower slot 118. To secure the strap 104, a first opposing free end 304 is fed (i.e. laced) through the first ladder lock buckle 208, and a second opposing free end 306 is fed through the second ladder lock buckle 302. The free ends 304, 306 are pulled in a direction opposite to the tubular body to secure under the scrotum as shown best in FIG.2. The ladder lock buckles 208, 302 described herein, can provide numerous adjustment lengths, so that the strap 104 can be used and secured to different scrotum sizes.

The strap 104 as used herein is a broad term that is inclusive of straps, belts, hook and loop fasteners or bands that can be formed from a variety of materials, such as elastic, cloth, nylon etc. The strap 104 can come in a variety of standard or non-standard widths (such as one-half inch, one inch, etc.).

While adjusting the length of the strap 104, the strap can remain slotted through the first continuous upper slot 110 and the first continuous lower slot 112, passing through the first support member 114, and similarly slotted through the second continuous upper slot 116 and the second continuous lower slot 118 passing through the second support member 120.

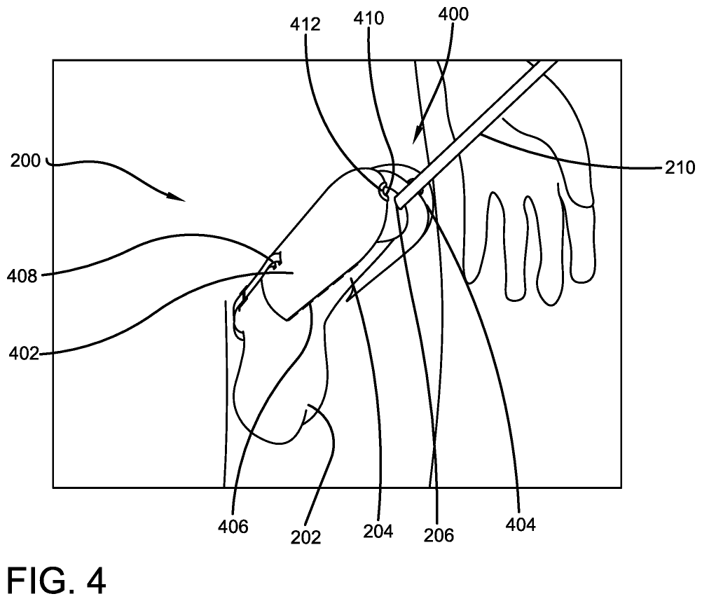

FIG. 4 illustrates a perspective view of another potential embodiment showing the male user wearing a glans guard device of the present invention in accordance with the disclosed architecture. The present embodiment of the glans guard device 400 includes a slotted body having a first tubular portion 402 and a second tubular portion 404 connected through the slit 406. The first tubular portion 402 and the second tubular portion 404 are selectively-detachable from each other along the slit 406. An opening 410 surrounded by a rubber grommet 412 is disposed at the top or dome section, thereby enabling a urinary catheter 210 to be inserted through the opening 410 into the urethra orifice 206 of the penis 204. The first tubular portion 402 and the second tubular portion 404 cover and conform to the shape of the penis 204 of the male user 200. For providing support to the tubular portions 402, 404, an extendable strap 408 connected to the first tubular portion 402 and the second tubular portion 404 wraps behind the scrotum 202 of the user 200. The strap 408 passes under the scrotum 202 (abutted against the perineum 207) to secure and stabilize the tubular portions 402, 404.

The attachment of the strap 408 and the adjustment through the buckles is similar to the other embodiments of the present invention. A pair of ladder lock buckles can function in a similar manner in the present embodiment as described earlier.

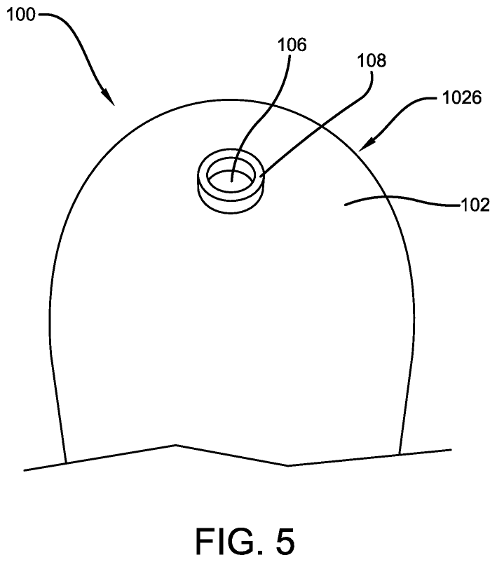

FIG. 5 illustrates a close view of the opening at a top of the tubular body of the glans guard of the present invention in accordance with the disclosed architecture. The tubular body 102 of the glans guard 100 includes an opening 106 surrounded by a rubber grommet 108 at the dome-shaped top end 1026 of the tubular body 102. The dome-shaped end 1026 is configured to conform to the glans of a patient, and the opening 106 enables a medical practitioner to insert a urinary catheter into the urethral opening/orifice.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “glans guard device”, “glans guard”, “glans protective device” and “device”, are interchangeable and refer to the glans guard device 100, 400 of the present invention.

Notwithstanding the forgoing, the glans guard device 100, 400 of the present invention can be of any suitable size and configuration as is known in the art without affecting the overall concept of the invention, provided that it accomplishes the above-stated objectives. One of ordinary skill in the art will appreciate that the size, configuration and material of the glans guard device 100, 400 as shown in the FIGS. are for illustrative purposes only, and that many other sizes and shapes of the glans guard device 100, 400 are well within the scope of the present disclosure. Although the dimensions of the glans guard device 100, 400 are important design parameters for user convenience, the glans guard device 100, 400 may be of any size that ensures optimal performance during use, and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A urinary catheterization protective device comprising:
a glans guard device, wherein said glans guard device comprises a tubular body having a dome shaped top end and an open bottom end, and further wherein said tubular body surrounds a majority of a penis of the user including a glans area and a foreskin area of the penis;
a strap attached to said bottom end of said tubular body, wherein said strap extends behind a scrotum of a user; and
an opening at a top end of said tubular body for extending a urinary catheter tube therethrough.

2. The urinary catheterization protective device of claim 1, wherein said tubular body comprises a first semi-circular section selectively secured to a second semi-circular section.

3. The urinary catheterization protective device of claim 1, wherein said strap is adjustable in length.

4. The urinary catheterization protective device of claim 1, wherein said opening is circumscribed by a flexible grommet.

5. The urinary catheterization protective device of claim 1, wherein said strap comprises a first ladder lock buckle for adjusting a length of said strap.

6. The urinary catheterization protective device of claim 5, wherein said strap comprises a second ladder lock buckle for adjusting said length of said strap.

7. The urinary catheterization protective device of claim 1, wherein said strap is elastic.

8. A urinary catheterization protective device comprising:
a glans guard device, wherein said glans guard device comprises a tubular body having a dome shaped top end and an open bottom end, and further wherein said tubular body surrounds a majority of a penis of the user including a glans area and a foreskin area of the penis;
an extendable strap attached to said bottom end of said tubular body, wherein said strap extends behind a scrotum of a user;
a first ladder lock buckle laced through said strap for securing an adjusted length of said strap;
an opening at a top end of said tubular body for extending a urinary catheter tube therethrough; and
a rubber grommet circumscribing said opening for frictionally retaining the catheter tube.

9. The urinary catheterization protective device of claim 8, wherein said tubular body comprises a first semi-circular section selectively secured to a second semi-circular section.

10. The urinary catheterization protective device of claim 8, wherein said strap comprises a second ladder lock buckle for securing said adjusted length of said strap.

11. The urinary catheterization protective device of claim 8, wherein said strap is elastic.

12. A method of protecting the glans of a male penis during a urinary catheterization, the method comprising the steps of:
providing a glans guard device, wherein said glans guard device comprises a tubular body having a dome shaped top end and an open bottom end;
surrounding a majority of the penis with said tubular body, wherein said tubular body covers at least a glans area and a foreskin area of the penis;
attaching a strap to said bottom end of said tubular body;
extending said strap around a scrotum, wherein the scrotum is positioned between said tubular body and said strap; and
adjusting a length of said strap.

13. The method of claim 12 further comprising a step of positioning the penis in a desired position for the urinary catheterization.

14. The method of claim 12, wherein said strap comprises a first ladder lock buckle for securing an adjusted length of said strap.

15. The method of claim 12, wherein said tubular body comprises an opening at a top end of said tubular body for extending a urinary catheter tube therethrough.

16. The method of claim 15, wherein said opening comprises a rubber grommet circumscribing said opening for frictionally retaining the catheter tube.

17. The method of claim 12, wherein said tubular body comprises a first semi-circular section selectively secured to a second semi-circular section.

18. The method of claim 14, wherein said strap comprises a second ladder lock buckle for securing said adjusted length of said strap.

19. The method of claim 12, wherein said strap is elastic.

20. The method of claim 14, wherein said first ladder lock buckle is laced through said strap.
